(19) United States
(12) Patent Application Publication
(10) Pub. No.: US 2022/0122728 A1
(43) Pub. Date: Apr. 21, 2022

(54) SYSTEM AND METHOD FOR BREATHING MONITORING AND MANAGEMENT

(71) Applicant: IBREVE LIMITED, Dublin (IE)

(72) Inventors: Jacob WAHL, Herrenberg (DE); Flavia WAHL, Herrenberg (DE)

(73) Assignee: IBREVE LIMITED, Dublin (IE)

(21) Appl. No.: 17/298,345

(22) PCT Filed: Dec. 2, 2019
(86) PCT No.: PCT/EP2019/083367

§ 371 Date: May 28, 2021

Related U.S. Application Data
(60) Provisional application No. 62/773,254, filed on Nov. 30, 2018.

(51) Int. Cl. G16H 50/20; G16H 40/67; G16H 50/30; G16H 10/60; G16H 50/70; A63B 71/06; A61B 5/00; A61B 5/08; A61B 5/16; A63B 24/00

(52) U.S. Cl. CPC G16H 50/20; G16H 20/10; G16H 50/30; G16H 10/60; G16H 50/70; A63B 71/0622; A61B 5/486; A61B 5/0816; A61B 5/4266; A61B 5/165; A61B 5/7275; A61B 5/4094; A61B 5/746; A61B 5/7405; A61B 5/7282; A61B 5/747; A61B 5/6823; A61B 5/7246; A63B 24/0062; A61B 5/4806; G16H 40/67

(57) ABSTRACT

A method and system for monitoring breathing comprising: detecting a respiratory pattern of a user; detecting an activity level of said user; determining a suitable actionable intervention recommendation based on the detected respiratory pattern and the detected activity level; presenting the actionable intervention recommendation to the user.

### CROSS-REFERENCE TO RELATED APPLICATION

This application claims the benefit of U.S. Provisional Application No. 62/773,254 filed 30 Nov. 2018 which is incorporated herein in its entirety by this reference.

### FIELD OF THE INVENTION

The present invention relates to digital health, well-being and medtech. In particular the present invention relates to method and system for monitoring breathing with applications in stress management, menopause management and epilepsy management.

### BACKGROUND TO THE INVENTION

One out of four adults suffers from stress, which puts them at higher risk of hypertension, heart diseases and other stress-related illnesses. Failing to cope with chronic stress can lead to physical as well as to mental health diseases.

The negative effects of stress are even more severe in patients suffering from a chronic disease. In addition to the everyday challenges that most people face, chronic illness adds additional stressors. Studies have shown that stress can worsen symptoms of chronic conditions and even trigger severe and sometimes life threatening reactions like an asthma attack, anxiety attack, epileptic seizure etc.

As social hurdles to get professional medical help are often high, drug abuse or overeating ‘comfort foods’ are easy go-to stress coping strategies. Current medical approaches to stress therapy are often centred around treatment and begin only after diagnosis of burnout or other stress-related diseases surface. Little is done in the ‘prevention phase’.

Although studies have proven that improving mental health increases the quality of life in patients with a chronic diseases, the emotional dimensions of these conditions are frequently overlooked when medical care is considered.

Recently, wearables have become popular for tracking fitness data. However, none focus on monitoring, predicting and improving stress levels and emotional well-being. Further, none can be seamlessly adapted to help patients of particular chronic diseases.

Regular trackers cannot fully track mental health. In particular, wearables cannot fully detect breathing patterns in motion and only detect vital signs like breathing rate and turn into standard step counters while in motion.

Regular trackers cannot be tailored to a particular disease and have often no medical and scientific validation.

Globally, cardiovascular diseases (CVDs) are the major cause of premature death and chronic disability, although the WHO states that most CVDs can be prevented by addressing physical and behavioural risk factors. Amongst women, heart disease is the number one killer, just before cancer. After the age of 50, nearly half of all deaths in women are due to some form of CVD. Cardiovascular risks in women are often poorly managed, especially during the menopausal transition when the probability of cardiovascular events increases significantly. A staggering 75% of women who seek medical attention for their symptoms is left untreated.

The most commonly used pharmacological menopause management method is hormone replacement therapy (HRT), which can give symptomatic relief from vasomotor symptoms like hot flushes. However, research suggests it can raise the risk of blood clots, stroke, and some cancers. Thus, HRT can't be recommended for the prevention of cardiovascular disease. Current femtech solutions mainly focus on pregnancy and menstrual cycle, while only a few solutions are targeting women's midlife health issues. While many mobile apps are often on the lower price spectrum, they often do not offer a biofeedback component. General wearables can be used to increase activity and relieve some symptoms like weight gain and stress, however they often lack medical accuracy and are not tailored to women's needs at this stage in their life.

Epilepsy is one of the most common disorders of the nervous system and affects more than 50 million people worldwide (WHO 2017). Currently there is no cure for epilepsy. In fact, anyone of us can have a seizure under certain conditions, for example through stress or sleep deprivation.

The unpredictable nature of seizures causes the largest burden for patients as it literally disrupts their lives. An epileptic seizure is usually characterized by uncontrolled shaking movements, which can involve the entire body and often the loss of consciousness. For people with epilepsy this can happen several times a day or adhoc out of the blue—even after a long period without a seizure. Seizures that lasts for more than a brief period of time are a medical emergency and can lead to death. Each year, more than 1 out of 1,000 people with epilepsy die from sudden unexpected death in epilepsy (SUDEP). If seizures are uncontrolled, the risk of SUDEP increases to more than 1 out of 150.

Predicting the onset and intensity of an epileptic seizure could solve one of the biggest burdens within epilepsy. Patients could enjoy greater freedom and confidence going about each day. It also provides the opportunity to intervene and respond ahead of time to lessen the severity of seizures and take safety measures ranging from taking anti-seizure drugs to getting into a safe position that prevents head injuries.

### SUMMARY OF THE INVENTION

In one aspect, the present invention provides a method for monitoring breathing comprising:

detecting a respiratory pattern of a user;
detecting an activity level of said user;
determining a suitable actionable intervention recommendation based on the detected respiratory pattern and the detected activity level;
presenting the actionable intervention recommendation to the user.

The respiration pattern preferably includes a respiration rate.

The actionable intervention recommendation may comprise a guided breathing exercise and presenting the actionable intervention recommendation to the user comprises running the guided breathing exercise on a user device.

The breathing exercise may be to exercise where the breathing matches the movement in the exercise.

The actionable intervention recommendation may comprise a guided meditation exercise and presenting the actionable intervention recommendation to the user may comprise running the guided meditation exercise on a user device.

The actionable intervention recommendation may comprise a guided game controlled by breathing or activity and presenting the actionable intervention recommendation to the user comprises running the guided game on a user device.

The method may further comprise tracking the user's performance during the exercise or game.

The method may further comprise detecting a respiratory pattern of a user and detecting an activity level of said user after the intervention recommendation has been performed. The method may then further comprise recording whether the actioned intervention helped.

The method may further comprise providing real-time feedback to the user. The feedback may be presented via a user device. The feedback may be presented via a worn device. Haptic feedback may be used. Feedback could be presented to multiple devices in a user group or class environment such as a yoga class.

The feedback may be in the form of a changeable coloured screen, wherein the colour reflects the current breathing pattern.

The method may further comprise calculating and storing personal baselines for the user through continuous monitoring of the respiratory pattern and activity level of said user.

The method may further comprise monitoring for variance from the personal baselines.

The method may further comprise generating a risk profile for the user.

The method may further comprise incorporating at least one of data from other devices, medical records, history or general population data in the risk profile.

The method may further comprise comparing the detected respiratory pattern and detected activity level to the risk profile.

The method may further comprise comparing the detected respiratory pattern and detected activity level with at least one of data from other devices, medical records, history or general population data.

The method may further comprise detecting the respiratory pattern and the activity level during mental exercise. The method may further comprise detecting the respiratory pattern and the activity level during physical exercise. The method may further comprise detecting the respiratory pattern and the activity level during sleep.

The method may further comprise correlating the detected respiratory pattern and the activity level during sleep with intervention recommendations actioned during the day.

The method may further comprise determining the type of breathing from the detected respiratory pattern. The type of breathing may be diaphragmatic breathing or chest breathing.

The method may further comprise detecting the posture of the user. The method may comprise notifying the user of bad posture and suggesting a change in posture. For example, a notification could be send if it is detected that the user has been still or sitting for an extended period, if it is detected that no deep breaths have taken place in a length of time, if it is detected that the breathing is fast or too fast, or if a prolonged holding of breath is detected.

The method may further comprise detecting for sweat on the user's skin.

The method may further comprise deriving the mood of the user based on the detected respiratory pattern and the detected activity level. The method may further comprise deriving the mood of the user based on the detected respiratory pattern and the detected activity level and user input. The method could include proposing an intervention based on a user input or request.

The method may further comprise comparing at least one of the respiratory pattern, activity level and mood to a cardiovascular risk profile.

The method may further comprise predicting a seizure based on the respiratory pattern. The method may further comprise generating an alert if a seizure is predicted. For example, the alert could inform a user that a seizure is likely in say the next 15 mins or other time period. The alert may be audible for the user, on a display or via a phone call.

The method may also monitor results from other sensors.

The method may further comprise comparing the detected activity level to a seizure pattern to diagnose that the user is experiencing a seizure. The method may further comprise comparing the detected respiratory pattern to a seizure pattern to diagnose that the user is experiencing a seizure.

The method may further comprise generating an alert if a seizure diagnoses is reached. The method may further comprise detecting the end of a seizure and generating a notification reflecting same. The method may further comprise tracking the respiratory pattern throughout a seizure. The method may further comprise detecting the body position of the user. The method may further comprise generating an alert if a seizure diagnoses is reached while a prone body position is detected.

The method may further comprise tracking the length of the seizure. The method may further comprise tracking the intensity of the seizure. The method may further comprise generating an alert if the length of the seizure exceeds a threshold and/or if the intensity of the seizure exceeds a threshold. The alert may comprise an audible alert. Generating an alert may comprise notifying a remote caregiver of the alert. Notifying a remote caregiver of the alert may comprise contacting emergency services via a phone call. The actionable intervention recommendation may comprise a recommendation for change in body position. This may be through a speaker to instruct a third part to turn the user during the seizure from the prone position. The actionable intervention recommendation may comprise a preventative behaviour suggestion. For example the user may be told to control their breathing to relax. The actionable intervention recommendation may comprise a recommendation for medication such as but not limited to anti-seizure medicine.

The respiratory pattern of the user may be detected using a wearable sensor worn on the chest. The activity level of the user may be detected using a wearable sensor worn on the chest. The body position of the user may be detected using a wearable sensor worn on the chest.

The present invention further provides a system for monitoring breathing comprising:

means for detecting a respiratory pattern of a user;
means for detecting an activity level of said user;
means for determining a suitable actionable intervention recommendation based on the detected respiratory pattern and the detected activity level; and
means for presenting the actionable intervention recommendation to the user.

The present invention further provides a data processing device comprising means for carrying out the aforementioned method. The present invention further provides a computer program comprising instructions which, when the program is executed by a computer, cause the computer to carry out the aforementioned method.

The present invention provides a self-care wearable solution that may focus on prevention and promotes healthy living. In this application, the wearable of the present invention can analyse breathing patterns in real-time and guide through clinically proven breathing and relaxation exercises. A further application of the invention is to help people suffering from stress, anxiety and chronic diseases.

By correlating breathing patterns, activity and stress level with machine learning in real time, the present invention can be used to prevent the rise of negative stress in an early phase. Breathing is one of the only parts of the autonomic nervous system that humans can actively control. Guided breathing exercises are clinically proven to significantly lower stress levels and trigger a rest and digest response by the parasympathetic nervous system.

The sensor device of the present invention may connect via Bluetooth to a mobile phone, but also works as a standalone device.

A machine learning algorithm may correlate breathing patterns, activity and optionally stress level and can trigger a subtle vibration directly on the sensor device or send a notification to a phone.

The present invention analyses respiratory patterns in real-time, enabling market applications for stress reduction, remote monitoring and chronic disease management.

The present invention can prevent stress in an early phase by correlating respiratory patterns, activity and stress triggers in real time. With the help of machine learning, the present invention can suggests a personalized treatment with clinically proven relaxation exercises.

The present invention enables better measurement, analysis and monitoring of health and stress indicators and acts as a platform to improve overall well-being and stress related diseases.

The wearable device of embodiments of the present invention can be attached to the body for measuring, monitoring, analysing and predicting health indicators and emotional well-being.

The personal coaching app and platform software may include stress management, relaxation exercises, guidance, tips, connection to other users (social network) and stakeholders like doctors, researchers etc. (see more stakeholders under use cases).

In the application of menopause management, the present invention may provide a digital therapeutics (DTx) solution that assists women in every step of their personal health care journey during and after their menopausal transition. It may be used to predict and prevent cardiovascular risk based on real-time bio signal analysis, suggest early interventions based on machine learning and achieves relief from the predominant menopausal symptoms through personalized physical and mental exercises. It may leverage gamification to encourage better habits by engaging and rewarding its users followed by interactive coaching and personal challenges.

The present invention's competitive advantages derive from the ability to personalize the solution based on bio signals and provide tailored non-invasive interventions exactly when needed most. The present invention may be used to offer relief from a wide array of common physical menopause symptoms (hot flushes, sleep problems and weight gain) as well as psychological problems (mood swings, stress and anxiety) through clinically proven exercises. On top, a woman's cardiovascular risk profile may be created and targeted interventions in form of exercises and knowledge mini pills may be provided.

The system of the present invention may comprise sensor technology to continuously analyse small variations in respiratory rate and breathing patterns. The system may include an Epileptic Seizure Prevention System (ESPS) deep tech solution integrated into a specifically designed and easy to use wearable for epilepsy patients. An ESPS machine learning algorithm may continuously analyse changes in respiratory pattern from the patient's personal baseline. Seizure probability and seizure intensity may be estimated and sent to the patient's smartphone (ESPS app). If desired an alert may be sent to the patient's caregiver (ESPS caregiver app).

The seizure itself may be monitored with motion sensors to track intensity, length and body position. If a high risk body position is detected, for example one related to sudden unexpected death in epilepsy patients (SUDEP), an immediate emergency call with the patient's exact location may be placed.

In general, an abnormal respiratory rate has been shown to be one of the most important predictors of serious health emergencies that lead to an intensive care unit admission. Focal seizures are frequently associated with significant respiratory abnormalities like increased respiratory rate, respiratory pauses or phases of apnea. Studies also indicate that respiration changes occur even earlier than other vital signs like heart rate.

Several methods for epilepsy treatment are being studied in recent years, including pharmacological, behavioural and alternative medicine therapies. None of them holistically integrates effective SUDEP prevention with an easy to use treatment approach by combining mental and physical therapeutic methods with a device based solution to accompany epilepsy patients in their everyday lives.

The solution of the present invention offers increased sensitivity compared to existing epilepsy management solutions. The present invention is able to track breathing patterns even during motion, walking, running etc.

Competitive advantages include the ability to monitor and differentiate between types of breathing and personalize relaxation exercises based on the user's breathing patterns.

Including the bra in the sensor mechanism ensures better results (due to the bra's fixed position) and user retention (as it builds on existing habits of wearing the bra).

The present invention may be personalized and adapts to the user's needs in real time, it may be predictive by learning from past situations and calculates personal baselines, thus it can prevent stress peaks and potential diseases caused by stress. Furthermore, it is participatory; it puts the user in control and empowers self-care.

### BRIEF DESCRIPTION OF THE DRAWINGS

Embodiments of the invention will be described, by way of example only, with reference to the accompanying drawings in which:

FIG. 1 is a block diagram of one embodiment of the system of the present invention;

FIG. 2 is a block diagram of one embodiment of the system of the present invention;

FIG. 3 is a block diagram showing possible impacts resulting from use of the present invention;

FIG. 4 is a block diagram of the hardware of one embodiment of the system of the present invention;

FIG. 5 shows a side view of one embodiment of sensor device and in use attached to a bra.

FIG. 6 shows a side view of another embodiment of sensor device and in use attached to a bra.

FIG. 7 shows example locations for a sensor device of the present invention.

FIG. 8 shows one embodiment of a sensor device attached to a chest belt, shown on and off the body.

FIG. 9 shows one embodiment of a sensor device attached to underwear.

FIG. 10 shows potential features and user cases.

FIG. 11 shows an exploded view of one embodiment of a wearable sensor device in accordance with one aspect of the present invention.

FIG. 12 shows a sensor device and app running on a user device in accordance with one embodiment of the system of the present invention.

FIG. 13 shows one embodiment of a wearable sensor device in accordance with one aspect of the present invention.

FIG. 14 shows additional embodiments of a wearable sensor device in accordance with one aspect of the present invention.

FIGS. 15 to 18 shows additional views of the sensor embodiments of FIG. 14.

FIG. 19 shows an example of feedback via an app run on a mobile phone in accordance with one aspect of the present invention.

### DETAILED DESCRIPTION OF THE DRAWINGS

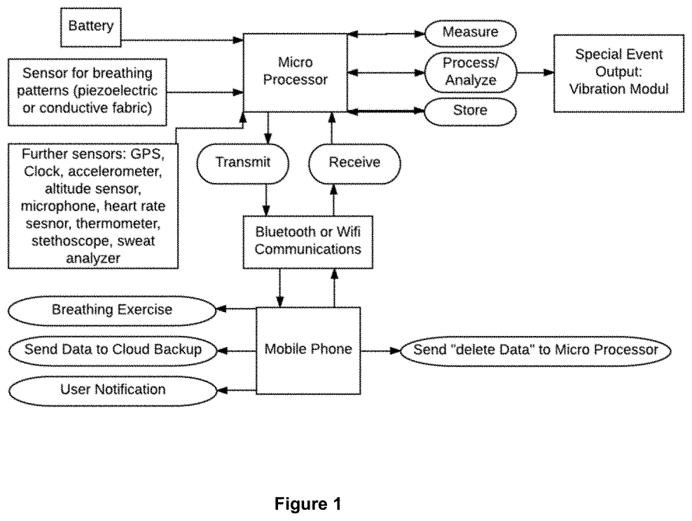

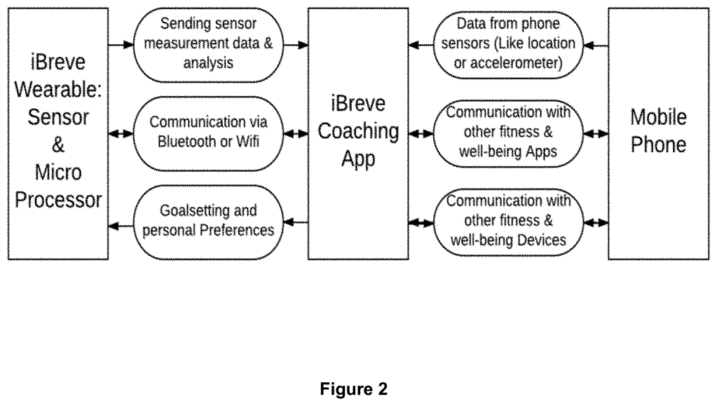

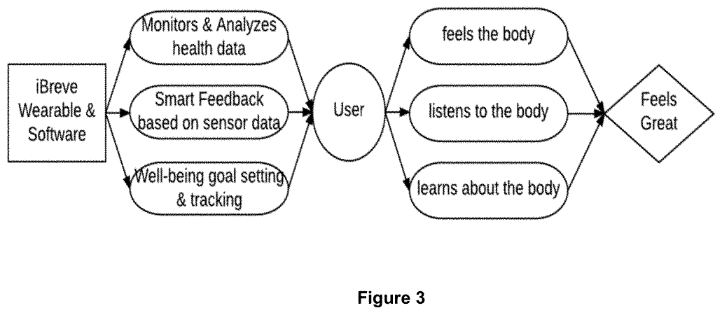

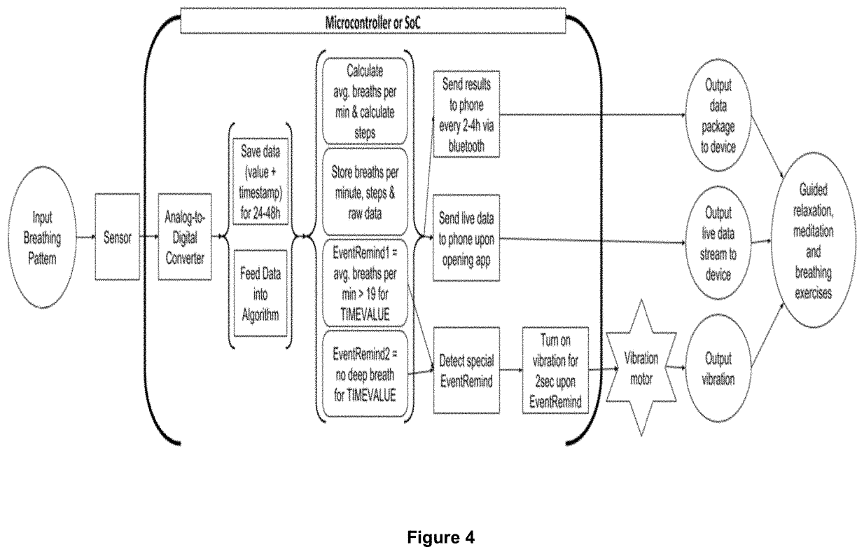

Embodiments of the system of the present invention are shown in FIGS. 1 and 2. In these embodiments, the system consists of a wearable sensor including processing capabilities, and a coaching app running software (machine learning) algorithms, typically on a mobile phone. The system may further comprise a cloud database.

The system of the present invention acts as a wearable and personal coach helping to cope with stress through enabling better measurement, monitoring and behaviour recommendations and uses machine learning based on health and stress indicators and their measurements like breathing patterns, chest expansion, stress level, heart rate, body temperature, lung health, menstrual cycle, pregnancy and pre-pregnancy indicators (e.g. ovulation timing), workout performance, readiness “state of fitness”, burned calories, PMS indicator, emotional tracking, biofeedback, biomarkers, in particular in extreme stress situations and for patients with chronic diseases, e.g. anxiety attack, asthma attack, seizure detection, prediction and prevention.

The sensor device of the present invention may comprise a combination of standard sensors (e.g. force sensor) and movement sensors (e.g. accelerometers, gyroscope) to measure breathing patterns, heartbeat and other vital signs and use algorithms to differentiate between chest movement/breathing/heartbeat and regular activity of the user.

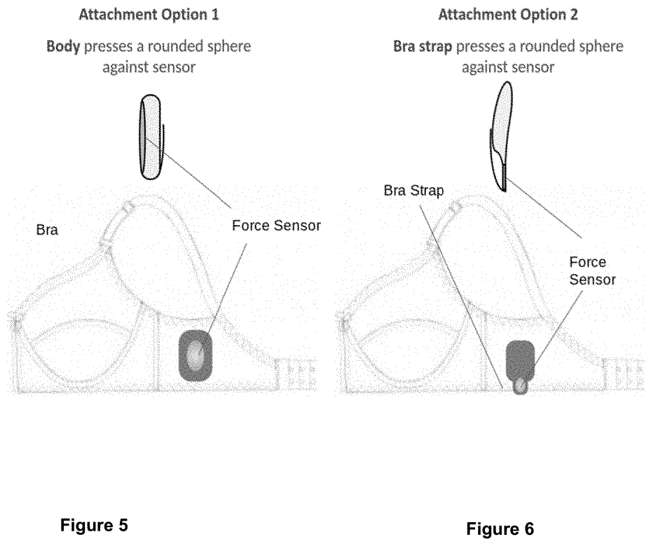

With reference to the embodiments shown in FIGS. 5 and 6, and in any embodiment where the sensor device is attached to a bra, the chest movement creates pressure against the bra strap, which extends and transfers it to a deformation material (e.g. a soft silicon) and is thus captured by a force sensor and an accelerometer and a gyroscope. The sensor signal is digitalized, recorded and analysed in real-time on the device, on the connected phone or in the cloud.

The system of the present invention can provide feedback, such as biofeedback and biomarkers, to suggest a relaxing breath or a break, and/or breathing and meditation exercises at the right moment (incl. vibrations, notifications) based on the above mentioned individual health and stress indicators and its machine learning algorithms. Further, it may aggregate and analyse the above measurements among users and users' activity data (including but not limited to users' phones and other wearable data like GPS, location, time, schedule, accelerometer, weather, altitude, microphone) and can be accessed by different stakeholders like doctors (see more under use cases).

By correlating breathing patterns, activity and stress level etc. with machine learning in real time, the present invention is able to prevent the rise of negative stress in an early phase and improves the user's well-being.

The present invention can prevent stress by identifying patterns leading to stress, like shallow breathing. It can effectively treats stress by guiding through clinically proven breathing and relaxation exercises. With real-time biofeedback and specifically developed breathing games, the present invention helps to improve the user's natural stress resilience long term.

The coaching app/software of the system can guide through personalized breathing and relaxation exercises based on clinically proven methods. It is possible to tailor content, training and programs to different target groups, patients, diseases, work environment etc. (see section on embodiments)

Users can access personalized subscriptions and relaxation packs from the iBreve cloud, can unlock features and personalize their user experience.

The system can give the right moment for a micro-meditation to instantly reduce stress. Its machine learning algorithm adapts to a user's lifestyle, behaviour and daily usage. Thus, can be tailored to people suffering from chronic diseases like epilepsy, respiratory diseases and mental diseases like general anxiety disorder

Prototypes have been tested in the lab and with pilots in the field. For example, prototypes were given to 40 yogis and asked them to follow a set sequence of yoga poses. The machine learning algorithm was able to distinguish between yoga beginners and teacher only by analysing the breathing curve.

Prototypes were also validated in an office setting, where they were given to 10 employees and monitored their regular work day. The fascinating thing here was that we could correlate certain tasks like opening the email inbox with certain patterns like holding the breath.

A system of the present invention is specifically designed to measure breathing continuously throughout the day and night. Most wearables on the market rely on accelerometers. The present invention may use a multi sensor technology, consisting of (but not limited to) a combination of a force sensor, stretch sensor (piezoelectric sensor or conductive fabric, QTC etc.) accelerometer, gyroscope and the elastic band of the bra or sports bra or textile.

As shown in FIGS. 5 and 6, the sensor device may be an add-on to the bra and make use of an everyday habit to achieve consistent usage. It provides tailored health insights in real time based on analysing breathing patterns, tracks daily activity and brings women's well-being to the next level.

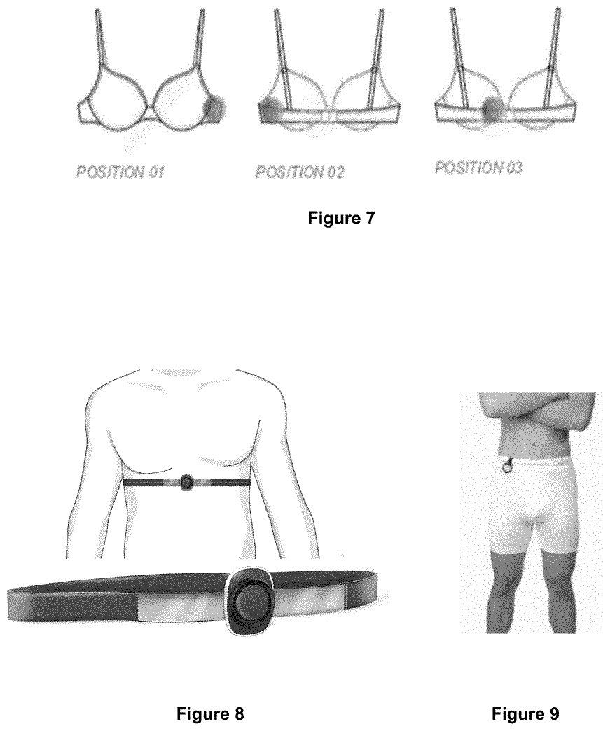

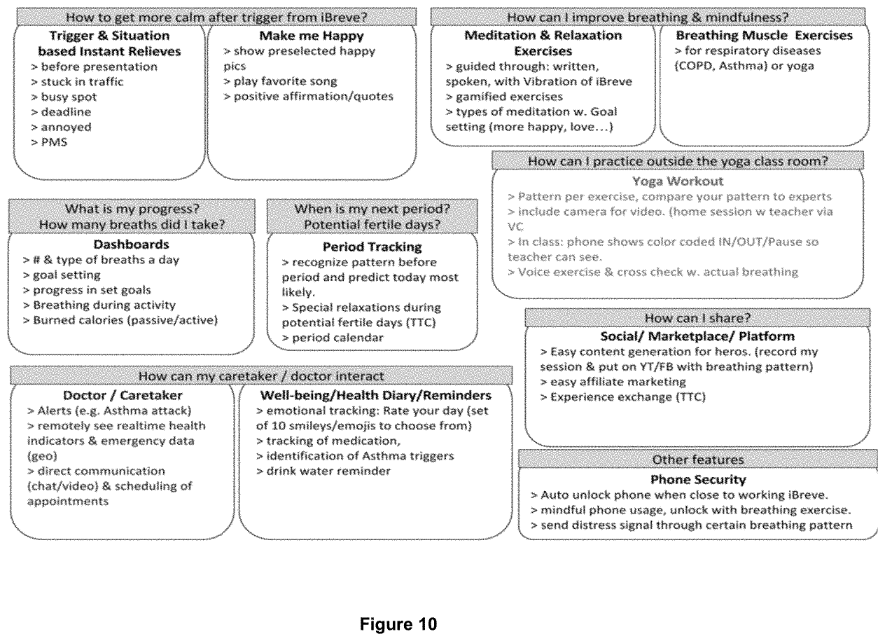

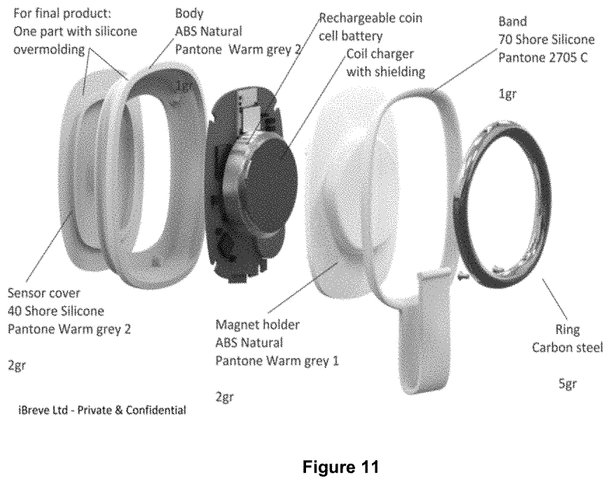

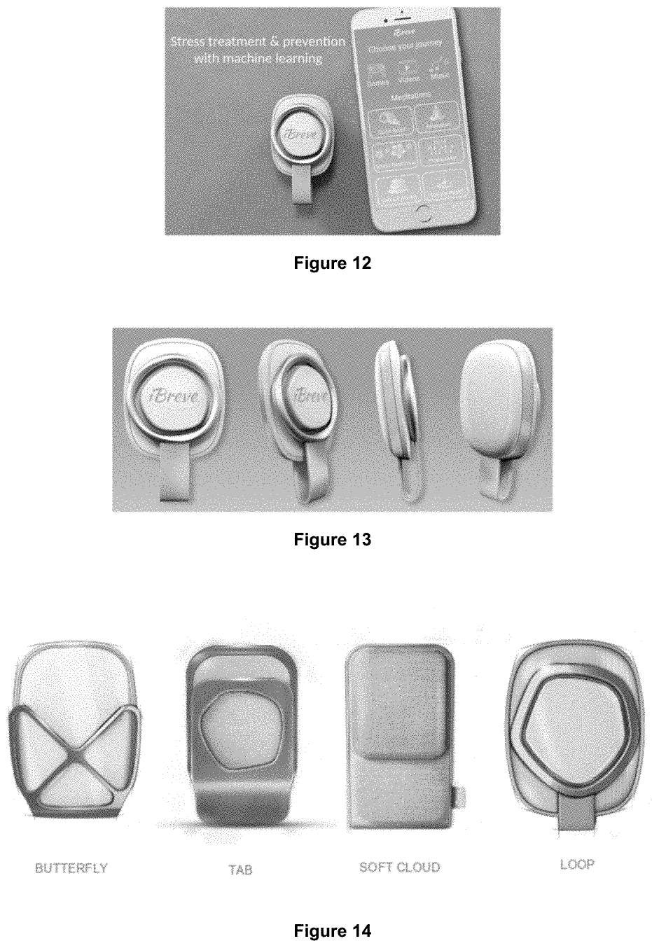

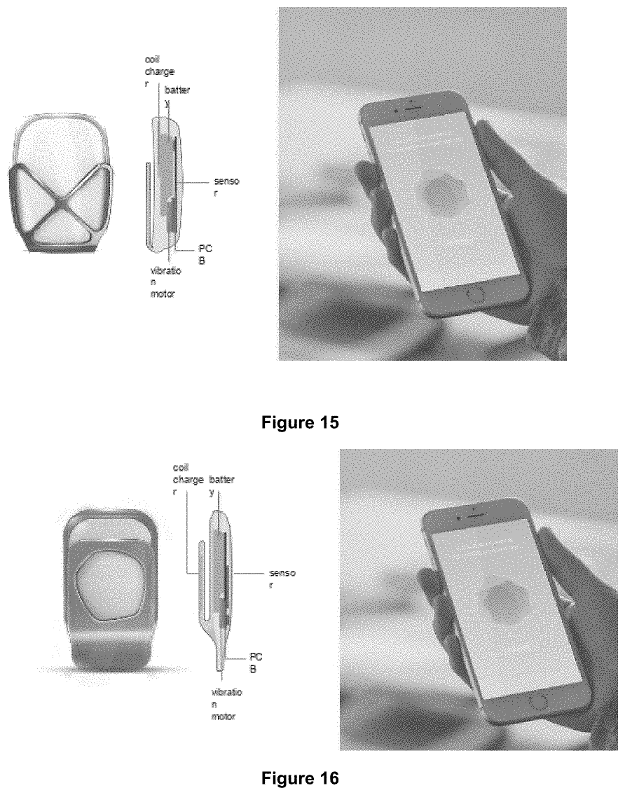

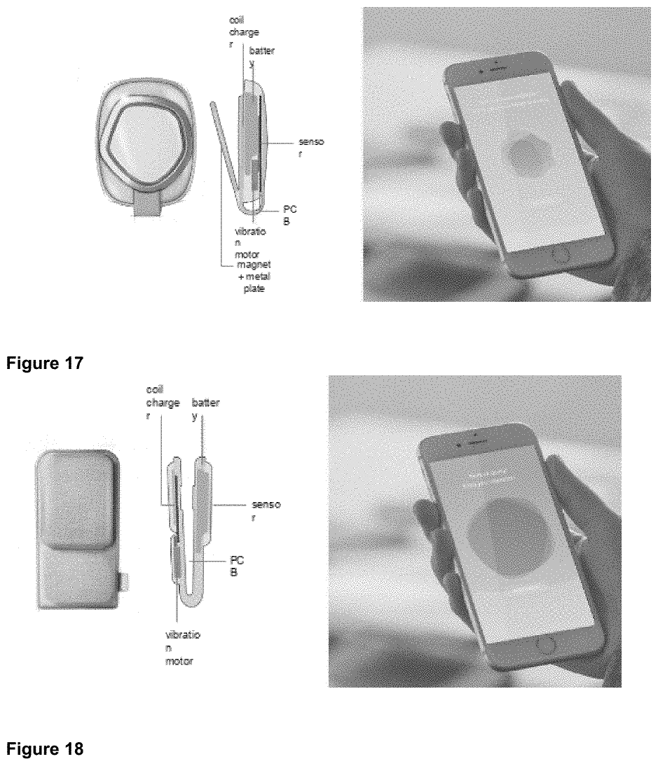

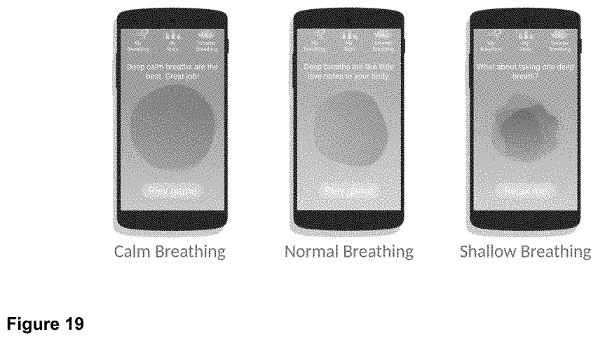

Alternatively, as shown in FIGS. 8 and 9, the sensor device can be attached to a chest belt, belt, trouser, underwear or any item able to hold the device against the skin. The device may alternatively be adhered directly to the skin. Thus the unobtrusive design may be specifically tailored to women and specifically tailored to men to ensure easy and daily usage. The system of the present invention is more than a physical fitness tracker, it has a strong impact on emotional well-being. It can be a personal coach which focuses on prevention and ability to alert the user before stress levels rise.

The inputs to the sensor device can include any of the following alone or in combination:
Breathing pattern
Steps counting (accelerometer)
Seated time
Double tap recognition
Type of breaths (Belly/diaphragmatic vs Chest)
Posture
Deep breath recognition
User input

Example hardware components are set out below:

### Computing and Connectivity

Microcontroller
Bluetooth Module
Wi-Fi module
Storage: Memory Card

### Sensors

Force Sensor (e.g. QTC, Piezo or similar)
Motion Sensor (accelerometer, gyroscope, magnetometer)
Custom silicone sensor part for working with any type of bra
Further sensors: GPS, clock, altitude sensor, microphone, heart rate sensor, body thermometer, stethoscope, sweat analyser

### Power Management

Wireless Charging Coil 18.3 mm*1.0 mm, well shielded
Coin cell, Rechargeable, 80 mAh, 3-3.7 V, compatible with selected charger and max height 2.5-3 mm
Lipo Charger Module
Voltage regulator
Custom Qi charging Station with coil of same diameter as receiver

### Notifications

Vibration Motor
LED

The sensing device is preferably connectable via Bluetooth 4.x/5 to a Virtual Coach app running on the user's mobile phone. The system may have wireless sync capability and wireless charging capability.

The storage environment for the embodiments of sensing device shown in the figures can have a temperature range of −10° C. to 50° C. The device is washing machine safe, water proof, has an operating environment both indoor and outdoor. When worn, the device is close to the body and touches the skin in a temperature range of 0° C. to 45° C., and is sweat proof.

Data Process capabilities include:
Read and save measurements from sensors
Analyse every X minutes according to preferences for “Special Event” (=stress indicator reaches certain level)
Give notification or vibration if “Special Event” happens (sensor device, phone, other wearables, computer)
Send data over BLE to phone or when user opens app and delete data on sensor device
Backup data from phone to cloud

By analysing the above mentioned health indicators, e.g. breathing patterns in real time, the system of the present invention can send smart alerts for motivation and behaviour change. The system of the present invention's tailored relaxation and breathing exercises give instant stress relief and long term health benefits through a more relaxed mind.

It is possible to provide useful insights with the app like setting well-being goals and data aggregation for preventive health alerts.

Examples of actionable intervention recommendations include:
Set relaxation goals
Tailored relaxation exercises
Counting breaths (Normal, shallow, deep chest, stomach)
Graph view of current breathing
Knows user's name
Menstrual cycle
Fitness tracking
breathing patterns, chest expansion, stress level, heart rate, body temperature, lung health, menstrual cycle, pregnancy and pre-pregnancy indicators (e.g. ovulation timing), workout performance, readiness “state of fitness”, burned calories, PMS indicator, emotional tracking.

Other stakeholders like doctors may have full access or limited access (user consent) to the recorded data, alerts, raw data, and/or analysis tools. It may be possible for other stakeholders to have a communication function to the user.

The present invention can help users to master moments of stress. The present invention can analyse individual breathing patterns and makes smart relaxation suggestions based on aggregated pattern recognition and users' activity data.

Example use cases include but are not limited to:
stress relief
yoga guidance
employee happiness and productivity increase
respiratory disease management
anxiety disorder treatment
burnout treatment
Office desk worker or employee
Fitness activities
Hospitals
Clinical Monitoring
Research and Field Tests
Insurance monitoring
Psychotherapie
Mindfulness based stress reduction (MBSR)
Cognitive behaviour therapy
Speaking and Presentations Training
Conferences
Performance Training
Military training and monitoring of soldiers and pilots
Professional Training (e.g. Leadership Training)
Women's health awareness programs (e.g. stress awareness)
Illness or chronic illness monitoring (e.g. Asthma, epilepsy)
Improve well-being and life quality of people suffering from chronic diseases
Epilepsy seizure detection, prediction and prevention
Epilepsy posture tracking during seizure (e.g. face up or down)
Extreme stress situation of patients with chronic diseases, for example Asthma attack, epileptic seizure, anxiety attack etc.

Benefits of the present invention include:
Reduces anxiety and depression
Lowers and stabilizes blood pressure
Increases energy levels
Leads to muscle relaxation
Decreases feelings of stress and overwhelm
Better sleep
Happier mind, more mindfulness
Decreasing unhealthy living and eating habits
Reduce stress and feel great
Live and breathe more healthy
Helps you to be calm and feel great
helps you get to know your body better
Simple to use
Effective
Get smart feedback exactly when you need it
Tailored to women's needs, tailored to men's needs
seamlessly fits into your life

As demonstrated above, the present invention helps to cope with stress by analysing respiratory patterns in real time. It offers a non-invasive preventative solution that empowers self-care and reduces stress in a simple, instant and natural way.

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1: A method for monitoring breathing comprising:
continuously detecting a respiratory pattern of a user via a wearable sensor;
detecting an activity level of said user at the time of detection of the respiratory pattern;
correlating the detected respiratory pattern and the detected activity level using a machine learning algorithm to determine a suitable actionable intervention recommendation, when a stress, seizure or cardiovascular event is predicted based on the correlated respiratory pattern and activity level; and
presenting the actionable intervention recommendation to the user.

2: The method of claim 1 wherein the actionable intervention recommendation comprises at least one of:
a guided breathing exercise and presenting the actionable intervention recommendation to the user comprises running the guided breathing exercise on a user device;
a guided meditation exercise and presenting the actionable intervention recommendation to the user comprises running the guided meditation exercise on a user device;
a guided game controlled by breathing or activity and presenting the actionable intervention recommendation to the user comprises running the guided game on a user device;
a recommendation for change in body position;
a preventative behaviour suggestion; or
a recommendation for medication.

3-5. (canceled)

6: The method of claim 1, further comprising detecting a respiratory pattern of a user and detecting an activity level of said user after the intervention recommendation has been performed to determine whether the actioned intervention helped.

7: The method of claim 1, further comprising providing real-time feedback to the user via a user device, or via a worn device.

8-9. (canceled)

10: The method of claim 7, wherein the feedback is in the form of a changeable coloured screen, wherein the colour reflects the current breathing pattern.

11: The method of claim 1, further comprising calculating and storing personal baselines for the user through continuous monitoring of the respiratory pattern and activity level of said user, and monitoring for variance from the personal baselines.

12. (canceled)

13: The method of claim 1, further comprising generating a risk profile for the user based on at least one of data from other devices, medical records, history or general population data in the risk profile.

14. (canceled)

15: The method of claim 11 wherein determining a suitable actionable intervention recommendation further comprises comparing the detected respiratory pattern and detected activity level to the risk profile.

16: The method of claim 1 further comprising comparing the detected respiratory pattern and detected activity level with at least one of data from other devices, medical records, history or general population data.

17-18. (canceled)

19: The method of claim 1 further comprising detecting the respiratory pattern and the activity level during at least one of: sleep or mental exercise or physical exercise.

20: The method of claim 19, further comprising correlating the detected respiratory pattern and the activity level during sleep with intervention recommendations actioned during the day.

21: The method of claim 1, wherein detecting a respiratory pattern of a user comprises determining the type of breathing.

22: The method of claim 21, wherein the type of breathing is diaphragmatic breathing or chest breathing.

23: The method of claim 1, further comprising detecting the posture of the user.

24: The method of claim 1, further comprising detecting for sweat on the user's skin.

25: The method of claim 1, further comprising deriving the mood of the user based on the detected respiratory pattern and the detected activity level.

26: The method of claim 1, further comprising deriving the mood of the user based on the detected respiratory pattern and the detected activity level and user input.

27: The method of claim 25 further comprising comparing at least one of the respiratory pattern, activity level and mood to a cardiovascular risk profile.

28: The method of claim 1, further comprising predicting a seizure based on the detected respiratory pattern.

29: The method of claim 28 further comprising generating an alert if a seizure is predicted.

30: The method of claim 1, further comprising comparing at least one of the detected respiratory pattern or the detected activity level to a seizure pattern to diagnose that the user is experiencing a seizure.

31: The method of claim 1, further comprising comparing the detected respiratory pattern to a seizure pattern to diagnose that the user is experiencing a seizure.

32: The method of claim 30 further comprising generating an alert if a seizure diagnoses is reached.

33: The method of claim 32 further comprising detecting the end of a seizure and generating a notification reflecting same.

34: The method of claim 31 further comprising tracking the respiratory pattern throughout a seizure.

35: The method of claim 1, further comprising detecting the body position of the user when a seizure is detected, and generating an alert if a seizure diagnoses is reached while a prone body position is detected.

36. (canceled)

37: The method of claim 28 further comprising tracking at least one of a length of the seizure or an intensity of the seizure; and generating an alert if the length of the seizure exceeds a threshold and/or if the intensity of the seizure exceeds a threshold.

38: The method of claim 31 further comprising tracking at least one of a length of the seizure or an intensity of the seizure.

39: The method of claim 38 further comprising generating an alert if the length of the seizure exceeds a threshold and/or if the intensity of the seizure exceeds a threshold.

40: The method of claim 29, wherein the alert comprises an audible alert.

41: The method claim 29, wherein generating an alert comprises notifying a remote caregiver of the alert.

42: The method of claim 41 wherein notifying a remote caregiver of the alert comprising contacting emergency services via a phone call.

43: The method of claim 1 wherein the actionable intervention recommendation comprises a recommendation for change in body position.

44: The method of claim 1, wherein the actionable intervention recommendation comprises a preventative behaviour suggestion.

45: The method of claim 1, wherein the actionable intervention recommendation comprises a recommendation for medication.

46: The method of claim 1, wherein the respiratory pattern of the user is detected using the wearable sensor worn on the chest.

47: The method of claim 1, wherein the activity level of the user is detected using the wearable sensor worn on the chest.

48: The method of claim 1, wherein the body position of the user is detected using the wearable sensor worn on the chest.

49: A system for monitoring breathing comprising:
means for detecting a respiratory pattern of a user via a wearable sensor;
means for detecting an activity level of said user at the time of detection of the respiratory pattern;
means for correlating the detected respiratory pattern and the detected activity level using a machine learning algorithm to determine a suitable actionable intervention recommendation, when a stress, seizure or cardiovascular event is predicted based on the detected respiratory pattern and the detected activity level; and
means for presenting the actionable intervention recommendation to the user.

50: The system of claim 49 wherein the actionable intervention recommendation comprises at least one of:
a guided breathing exercise and the means for presenting the actionable intervention recommendation to the user comprises means for running the guided breathing exercise on a user device; or
a guided meditation exercise and the means for presenting the actionable intervention recommendation to the user comprises means for running the guided meditation exercise on a user device; or
a guided game controlled by breathing or activity and the means for presenting the actionable intervention recommendation to the user comprises means for running the guided game on a user device.

51. (canceled)

52. (canceled)

53: The system of claim 50, further comprising means for tracking the user's performance during the exercise or game.

54: The system of claim 49, further comprising means for detecting a respiratory pattern of a user and means for detecting an activity level of said user after the intervention recommendation has been performed.

55: The system of claim 49, further comprising means for providing real-time feedback to the user.

56: The system of claim 55 wherein the feedback is presented via a user device.

57: The system of claim 56 wherein the feedback is presented via a worn device.

58: The system of claim 56 wherein the feedback is in the form of a changeable coloured screen, wherein the colour reflects the current breathing pattern.

59: The system of claim 49, further comprising means for calculating and storing personal baselines for the user through continuous monitoring of the respiratory pattern and activity level of said user.

60: The system of claim 59 further comprising means for monitoring for variance from the personal baselines.

61: The system of claim 49, further comprising means for generating a risk profile for the user.

62: The system of claim 61 further comprising means for incorporating at least one of data from other devices, medical records, history or general population data in the risk profile.

63: The system of claim 61 further comprising means for comparing the detected respiratory pattern and detected activity level to the risk profile.

64: The system of claim 49 further comprising means for comparing the detected respiratory pattern and detected activity level with at least one of data from other devices, medical records, history or general population data.

65: The system of claim 49 further comprising mean for detecting the respiratory pattern and the activity level during mental exercise.

66: The system of claim 49 further comprising means for detecting the respiratory pattern and the activity level during physical exercise.

67: The system of claim 49 further comprising detecting the respiratory pattern and the activity level during sleep.

68: The system of claim 67, further comprising means for correlating the detected respiratory pattern and the activity level during sleep with intervention recommendations actioned during the day.

69: The system of claim 49, further comprising means for determining the type of breathing from the detected respiratory pattern.

70: The system of claim 69, wherein the type of breathing is diaphragmatic breathing or chest breathing.

71: The system of claim 49, further comprising means for detecting the posture of the user.

72: The system of claim 49, further comprising means for detecting for sweat on the user's skin.

73: The system of claim 49, further comprising means for deriving the mood of the user based on the detected respiratory pattern and the detected activity level.

74: The system of claim 49, further comprising means for deriving the mood of the user based on the detected respiratory pattern and the detected activity level and user input.

75: The system of claim 73 further comprising means for comparing at least one of the respiratory pattern, activity level and mood to a cardiovascular risk profile.

76: The system of claim 49, further comprising means for predicting a seizure based on the respiratory pattern.

77: The system of claim 76 further comprising means for generating an alert if a seizure is predicted.

78: The system of claim 49, further comprising means for comparing the detected activity level to a seizure pattern to diagnose that the user is experiencing a seizure.

79: The system of claim 49, further comprising means for comparing the detected respiratory pattern to a seizure pattern to diagnose that the user is experiencing a seizure.

80: The system of claim 78 further comprising means for generating an alert if a seizure diagnoses is reached.

81: The system of claim 80 further comprising means for detecting the end of a seizure and means for generating a notification reflecting same.

82: The system of claim 79 further comprising means for tracking the respiratory pattern throughout a seizure.

83: The system of claim 49, further comprising means for detecting the body position of the user.

84: The system of claim 83 further comprising means for generating an alert if a seizure diagnoses is reached while a prone body position is detected.

85: The system of claim 78 further comprising: means for tracking the length of the seizure, or means for tracking the intensity of the seizure.

86: The system of claim 79 further comprising: means for tracking the length of the seizure, or means for tracking the intensity of the seizure.

87: The system of claim 85 further comprising means for generating an alert if the length of the seizure exceeds a threshold and/or if the intensity of the seizure exceeds a threshold.

88: The system of claim 77 wherein the alert comprises an audible alert.

89: The system of claim 77 wherein the means for generating an alert comprises means for notifying a remote caregiver of the alert.

90: The system of claim 89 wherein the means for notifying a remote caregiver of the alert comprising means for contacting emergency services via a phone call.

91: The system of claim 49 wherein the actionable intervention recommendation comprises: a recommendation for change in body position or a preventative behaviour suggestion or a recommendation for medication.

92-93. (canceled)

94: The system of claim 49, wherein the means for detecting the respiratory pattern of the user comprises a wearable sensor worn on the chest.

95: The system of claim 49, wherein the means for detecting the activity level of the user comprises a wearable sensor worn on the chest.

96: The system of claim 49, wherein the means for detecting the body position of the user comprises a wearable sensor worn on the chest.

97: A data processing device comprising means for carrying out the method of claim 1.

98: A computer program product comprising a non-transitory computer-readable medium including instructions adapted to be executed by a computer to implement a method for monitoring breathing, the method comprising:
continuously detecting a respiratory pattern of a user via a wearable sensor;
detecting an activity level of said user at the time of detection of the respiratory pattern;
correlating the detected respiratory pattern and the detected activity level using a machine learning algorithm to determine a suitable actionable intervention recommendation, when a stress, seizure or cardiovascular event is predicted based on the correlated respiratory pattern and activity level; and
presenting the actionable intervention recommendation to the user.

99: The computer program product of claim 98, wherein the actionable intervention recommendation comprises at least one of:
a guided breathing exercise and presenting the actionable intervention recommendation to the user comprises running the guided breathing exercise on a user device; or
a guided meditation exercise and presenting the actionable intervention recommendation to the user comprises running the guided meditation exercise on a user device; or
a guided game controlled by breathing or activity and presenting the actionable intervention recommendation to the user comprises running the guided game on a user device; or
a recommendation for change in body position; or
a preventative behaviour suggestion; or
a recommendation for medication.

100: The computer program product of claim 98, in which the method further comprises: detecting a respiratory pattern of a user and detecting an activity level of said user after the intervention recommendation has been performed to determine whether the actioned intervention helped.

101: The computer program product of claim 98, in which the method further comprises: providing real-time feedback to the user via a user device, or via a worn device.

102: The computer program product of claim 101, wherein the feedback is in the form of a changeable coloured screen, wherein the colour reflects the current breathing pattern.

103: The computer program product of claim 98, in which the method further comprises: calculating and storing personal baselines for the user through continuous monitoring of the respiratory pattern and activity level of said user, and monitoring for variance from the personal baselines.

104: The computer program product of claim 98, in which the method further comprises: generating a risk profile for the user based on at least one of data from other devices, medical records, history or general population data in the risk profile.

105: The computer program product of claim 103, wherein determining a suitable actionable intervention recommendation further comprises comparing the detected respiratory pattern and detected activity level to the risk profile.

106: The computer program product of claim 98, wherein detecting a respiratory pattern of a user comprises determining the type of breathing.

107: The computer program product of claim 98, in which the method further comprises: predicting a seizure based on the detected respiratory pattern and generating an alert if a seizure is predicted.

108: The computer program product of claim 98, in which the method further comprises: comparing at least one of the detected respiratory pattern or the detected activity level to a seizure pattern to diagnose that the user is experiencing a seizure.

109: The computer program product of claim 98, in which the method further comprises: detecting the body position of the user when a seizure is detected, and generating an alert if a seizure diagnoses is reached while a prone body position is detected.

110: The computer program product of claim 107, in which the method further comprises: tracking at least one of a length of the seizure or an intensity of the seizure; and generating an alert if the length of the seizure exceeds a threshold and/or if the intensity of the seizure exceeds a threshold.

111: The computer program product of claim 108, in which the method further comprises: tracking at least one of a length of the seizure or an intensity of the seizure; and generating an alert if the length of the seizure exceeds a threshold and/or if the intensity of the seizure exceeds a threshold.

112: The computer program product of claim 109, in which the method further comprises: tracking at least one of a length of the seizure or an intensity of the seizure; and generating an alert if the length of the seizure exceeds a threshold and/or if the intensity of the seizure exceeds a threshold.

113: The method of claim 30 further comprising tracking at least one of a length of the seizure or an intensity of the seizure; and generating an alert if the length of the seizure exceeds a threshold and/or if the intensity of the seizure exceeds a threshold.

114: The method of claim 35 further comprising tracking at least one of a length of the seizure or an intensity of the seizure; and generating an alert if the length of the seizure exceeds a threshold and/or if the intensity of the seizure exceeds a threshold.

115: The method of claim 26 further comprising comparing at least one of the respiratory pattern, activity level and mood to a cardiovascular risk profile.

116: The method of claim 31 further comprising generating an alert if a seizure diagnoses is reached.

117: The method of claim 116 further comprising detecting the end of a seizure and generating a notification reflecting same.

118: The method of claim 32, wherein the alert comprises an audible alert.

119: The method of claim 116, wherein the alert comprises an audible alert.

120: The method of claim 39, wherein the alert comprises an audible alert.

121: The method of claim 32, wherein generating an alert comprises notifying a remote caregiver of the alert.

122: The method of claim 39, wherein generating an alert comprises notifying a remote caregiver of the alert.

123: The method of claim 40, wherein generating an alert comprises notifying a remote caregiver of the alert.

124: The method of claim 116, wherein generating an alert comprises notifying a remote caregiver of the alert.

125: The method of claim 118, wherein generating an alert comprises notifying a remote caregiver of the alert.

126: The method of claim 119, wherein generating an alert comprises notifying a remote caregiver of the alert.

127: The method of claim 120, wherein generating an alert comprises notifying a remote caregiver of the alert.

128: The system of claim 79 further comprising means for generating an alert if a seizure diagnoses is reached.
